(19) United States
(12) Patent Application Publication
(10) Pub. No.: US 2023/0053766 A1
(43) Pub. Date: Feb. 23, 2023

(54) SYSTEM FOR TREATMENT PLANNING ENVIRONMENTS

(71) Applicant: uLab Systems, Inc., Memphis, TN (US)

(72) Inventors: Henry CAO, San Jose, CA (US); Huafeng WEN, Redwood Shores, CA (US); Surya SARVA, Fremont, CA (US); Andrew LIU, Burnaby (CA)

(73) Assignee: uLab Systems, Inc., Memphis, TN (US)

(21) Appl. No.: 17/664,854

(22) Filed: May 24, 2022

Related U.S. Application Data
(60) Provisional application No. 63/235,317, filed on Aug. 20, 2021.

(51) Int. Cl. A61C 7/00; A61C 9/00; G16H 80/00

(52) U.S. Cl. CPC A61C 7/002; A61C 9/0046; G16H 80/00

(57) ABSTRACT

Systems for treatment planning environments are disclosed where a method of collaborating on a treatment plan for correcting one or more teeth of a subject may include digitally receiving intra-oral scan data of one or more teeth of the subject where the one or more teeth are in an initial position, synchronizing a virtual treatment planning environment between a first device of at least one practitioner and a second device of at least one technician, and displaying a pre-setup design of the intra-oral scan data within the treatment planning environment. The pre-setup design is revised based upon input received from the practitioner for correcting one or more malocclusions and one or more intermediate steps of the treatment plan is created from the initial position to a final position of the teeth within the treatment planning environment. The treatment plan may be finalized by the practitioner within the treatment planning environment

### CROSS-REFERENCE TO RELATED APPLICATIONS

This application claims the benefit of U.S. Provisional Patent Application No. 63/235,317, filed Aug. 20, 2021, the content of which is incorporated herein by reference in its entirety.

### FIELD OF THE INVENTION

The present invention relates to methods and apparatus for computerized orthodontics treatment planning. More particularly, the present invention relates to methods and apparatus for planning orthodontic treatments by facilitating real-time collaboration between orthodontic practitioners and technicians within a virtual treatment planning environment.

### BACKGROUND OF THE INVENTION

Orthodontics is a specialty of dentistry that is concerned with the study and treatment of malocclusions which can result from tooth irregularities, disproportionate facial skeleton relationships, or both. Orthodontics treats malocclusion through the displacement of teeth via bony remodeling and control and modification of facial growth.

This process has been traditionally accomplished by using static mechanical force to induce bone remodeling, thereby enabling teeth to move. In this approach, braces having an archwire interface with brackets are affixed to each tooth. As the teeth respond to the pressure applied via the archwire by shifting their positions, the wires are again tightened to apply additional pressure. Other treatments for treating malocclusions may involve the use of orthodontic aligners which are fabricated to be placed upon the dentition and which incrementally shift the position of one or more of the teeth over time. The orthodontic practitioner may develop a treatment plan for moving the one or more teeth using treatment planning software such as one developed by uLab Systems, Inc. (San Mateo, Calif.). Once a treatment plan has been developed by the practitioner, they may also fabricate one or more of the aligners onsite using, for example, additive printing technologies.

Oftentimes, the practitioner may desire the development of a treatment plan in collaboration with a third party which can perform many of the details in treatment planning and fabrication. Accordingly, there exists a need for an efficient and effective process for planning the orthodontic treatment of a patient.

### SUMMARY OF THE INVENTION

In preparing a treatment plan, a practitioner may prepare a treatment plan for correcting the malocclusions on their own or the practitioner may collaborate with a treatment plan designer/technician trained in facilitating the treatment planning with the practitioner. A software structure which is able to join the practitioner and the treatment plan designer/technician in a treatment planning environment may be incorporated into a treatment planning software to enable efficient collaboration in real-time. The treatment planning software may be implemented upon various devices or interfaces having a processor such as a computer, tablet, smartphone, etc.

The practitioner is able to collaborate in real-time with the treatment plan designer/technician through a treatment planning environment (TPE) module integrated into the treatment planning software. While the practitioner is logged into the TPE of the treatment planning software, the practitioner may electronically submit intra-oral scan data of the patient's dentition obtained by the practitioner or another party. This intra-oral scan data may be uploaded to the TPE where the intra-oral scan data may be accessed by the treatment plan designer/technician (either in real-time simultaneously with the practitioner or at a later time) who may correct for any discrepancies in the dentition by forming an initial treatment plan for review by the practitioner. The treatment plan designer/technician may process the scan data using the treatment planning software to perform various other operations such as patching holes that may be present in the scan file, fixing a bite between the upper and lower teeth, segmenting the teeth in the scan data, identifying features such as the FACC line and ridge line, etc. Once the treatment plan designer/technician has completed correcting the scan data, the treatment plan designer/technician may save the revised scan data which is then saved on the practitioner's computer as well.

In the event that the treatment plan designer/technician accesses and corrects for discrepancies asynchronously from the practitioner, both the treatment plan designer/technician and practitioner may schedule a treatment planning session between the two over the TPE to discuss the initial treatment plan. One or more available time slots may appear on the TPE for both the treatment plan designer/technician and practitioner allowing for both parties to schedule a common time slot.

At the scheduled time, both the treatment plan designer/technician and practitioner may access the TPE simultaneously within the treatment planning software and collaborate in real-time over the TPE to adjust any number of aspects of the treatment plan. The treatment plan designer/technician or practitioner may host the session in the TPE and they may initiate the display of the data including the arches of the patient's dentition. The practitioner may instruct the treatment plan designer/technician how to set up the arches for the patient's final positioning for their teeth. The collaboration may include not only a display of the scan data upon the treatment plan designer/technician's screen and the practitioner's screen of their local computing device, but may also include voice communication over the TPE and optionally video communication of the treatment plan designer/technician and practitioner as well which may be transmitted through the internet and/or cloud storage service. As the practitioner instructs the treatment plan designer/technician, the treatment plan designer/technician may implement the desired changes and alterations using the treatment planning software in real-time showing the changes reflected to both screens of the respective computing devices of the treatment plan designer/technician and practitioner within the TPE so that the practitioner may visually see exactly what changes are being made to the digital model of the dentition for the treatment plan.

Once the treatment plan has been suitably adjusted in real time, the practitioner may approve the final treatment plan over the TPE using an approval processes (such as pressing of a button in lieu of a signature) and the final treatment plan may be implemented by the practitioner upon the patient's dentition including the ordering of any orthodontic prostheses used to carry out the treatment plan. The TPE may then be closed at the end of this process.

There are additional functions which may be optionally incorporated. In another variation, after the treatment plan has been approved, the practitioner can choose to send a link to the patient (e.g., via email, notification through an app on their tablet or smartphone, or through another communication method). Once the patient receives this link, they may access the results of the treatment plan showing, for instance, the movement of teeth from the initial position to a final position.

Additionally and/or alternatively, the TPE may be developed as a platform utilizing artificial intelligence algorithms to train the computers on the back end to understand the commands (verbal or textual) of the practitioner to implement their clinical demands so that the computer itself may implement any changes rather than a treatment plan designer/technician.

As the TPE may reside as a virtual module integrated with the treatment planning software, the practitioner may interact with the TPE through the internet and/or cloud storage service and via their local computing device with textual or voice feedback in response to 3D visual feedback and/or voice feedback from the treatment plan designer/technician who may similarly provide voice feedback to the practitioner via the TPE as well as receive the voice feedback from the practitioner via the TPE.

The treatment plan designer/technician may implement any number of treatment planning operations using the treatment planning software in response to the feedback from the practitioner received via the TPE. The treatment planning data as modified by the planning operations may be transmitted to the internet or cloud storage service which may then transmit the information through the TPE for presentation to the practitioner or directly to a local computing device of the practitioner for review.

In one variation, a method of collaborating on a treatment plan for correcting one or more teeth of a subject may generally comprise digitally receiving intra-oral scan data of one or more teeth of the subject where the one or more teeth are in an initial position, synchronizing a virtual treatment planning environment between a first device of at least one practitioner and a second device of at least one technician, and displaying a pre-setup design of the intra-oral scan data within the treatment planning environment, wherein the pre-setup design incorporates one or more corrections of the intra-oral scan data. The pre-setup design may be revised based upon input received from the at least one practitioner within the treatment planning environment for correcting one or more malocclusions and one or more intermediate steps of the treatment plan may be created from the initial position to a final position of the one or more teeth within the treatment planning environment. The treatment plan may be finalized by the practitioner within the treatment planning environment.

In another variation, yet another method for collaborating on a treatment plan for correcting one or more teeth of a subject may generally comprise digitally receiving intra-oral scan data of one or more teeth of the subject from at least one practitioner, wherein the one or more teeth are in an initial position, confirming a request received from the at least one practitioner via a virtual treatment planning environment to collaborate with at least one technician, and synchronizing a pre-setup design of the intra-oral scan data with a first device of the at least one practitioner with a second device of the at least one technician, wherein the pre-setup design incorporates one or more corrections provided by the at least one technician of the intra-oral scan data. The pre-setup design of the intra-oral scan data may be displayed within the treatment planning environment to the at least one practitioner and the at least one technician and the pre-setup design may be revised based upon input received from the at least one practitioner within the treatment planning environment for correcting one or more malocclusions. One or more intermediate steps of the treatment plan may be created from the initial position to a final position of the one or more teeth within the treatment planning environment to complete the treatment plan.

### BRIEF DESCRIPTION OF THE DRAWINGS

The following description of the figures of specific embodiments of the invention is merely exemplary in nature and is not intended to limit the present teachings, their application or uses. Throughout the drawings, corresponding reference numerals indicate like or corresponding parts and features.

FIG. 1A shows an exemplary process for scanning the patient's dentition, treatment planning, and then fabricating one or more aligners for effecting patient treatment.

FIG. 1B shows an example of a flow diagram illustrating how an initial treatment planning may be reassessed and additional treatment options may be generated or considered during additional treatment planning.

FIG. 2 shows a flow diagram of one exemplary method for a tooth modeling system.

FIG. 3A shows a flow diagram generally illustrates how the practitioner is able to collaborate in real-time with the treatment plan designer/technician through a treatment planning environment module integrated into the treatment planning software.

FIG. 3B shows a schematic illustrating both the treatment plan designer/technician and practitioner accessing the TPE simultaneously within the treatment planning software to collaborate in real-time over the TPE.

FIG. 4 shows a schematic diagram illustrating further details of how the practitioner and treatment plan designer/technician can facilitate the real-time collaboration through the TPE.

FIG. 5 shows a flow diagram another variation of the workflow for the practitioner and treatment plan designer/technician collaborating through the TPE.

### DETAILED DESCRIPTION OF THE INVENTION

The present invention will be described with respect to particular embodiments but the invention is not limited thereto but only by the claims. Any reference signs in the claims shall not be construed as limiting the scope thereof.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” when referring to recited members, elements or method steps also include embodiments which “consist of” said recited members, elements or method steps.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In treating a patient to correct for one or more conditions with their dentition, the steps of digitally scanning the patient's dentition, planning the treatment, and/or optionally fabricating the treatment devices, such as aligners to correct positioning of one or more teeth, may be performed directly at the provider's office.

With treatment planning software, a treatment plan using aligners, brackets, etc. may be used to correct for any number of malocclusions with a patient's teeth. Particular treatment planning processes are described in further detail in U.S. Pat. Nos. 10,624,717; 10,335,250; 10,631,953; 10,357,336; 10,357,342; 10,588,723; 10,548,690, as well as U.S. Pat. Pubs. 2017/0100208; 2019/0321135; 2020/0205936; 2019/0343602; 2020/0170762; 2018/0078343; 2018/0078344; 2018/0078335; 2020/0146775. The details of these references are incorporated herein by reference in their entirety and for any purpose.

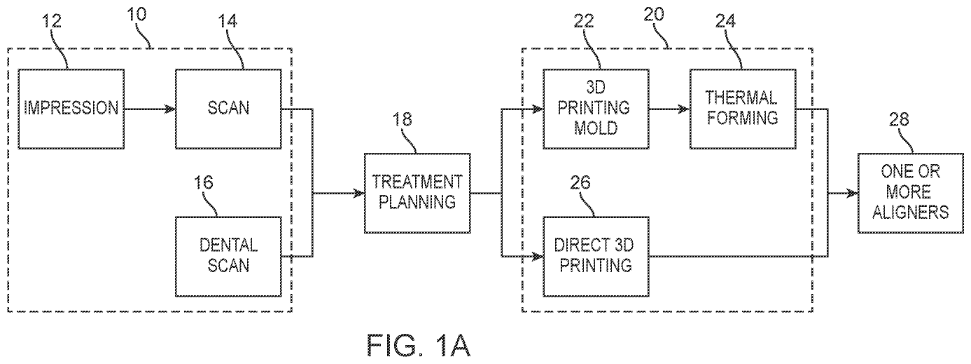

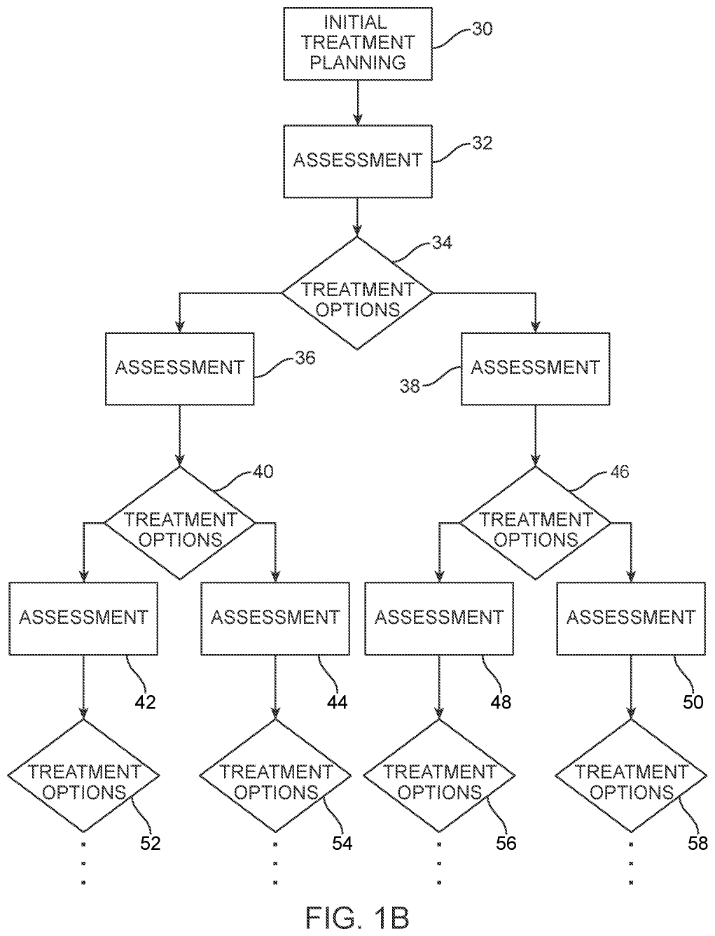

In preparation for using a treatment planning software, a general overview of the process for obtaining digital images of the dentition, treatment planning, and optionally fabricating the orthodontic prostheses are shown in the exemplary process of FIG. 1A. The step of scanning the patient's dentition 10 may be performed using a number of different processes. With the resulting digital images of the patient's dentition, the treatment planning 18 to correct for the positioning, misalignment, malocclusion, etc. of any one or more teeth may be performed using any of the processes described herein. Conventional treatment planning typically creates an entire treatment plan starting with the initial positioning of the patient's dentition and formulating a treatment based on a stepped realignment of the dentition. This stepped realignment is then used to create an entire array of aligners starting with an initial aligner and ending with a final aligner for use through the entire treatment process.

However, the treatment planning 18 and fabrication process 20 described herein may be performed on a variable treatment path. That is, while the initial treatment planning 18 may be based upon the initial positioning of the patient's dentition, the step-by-step process for subsequent treatments is variable such that the final treatment step is not pre-determined. Rather, the alignment of the dentition is determined at intermediate steps in which the patient may (or may not) come back to the practitioner's office for reassessments and potentially new scans and aligners for one or more intermediate treatment steps. In this manner, additional aligners or other treatment processes may be created during each visit to the practitioner by the patient. Hence, the entire treatment process is created as the treatment progresses thus leading to the treatment planning 18 and fabrication process 20 as an iterative process rather than an entire treatment sequence pre-determined at the treatment outset.

An example is shown in the flow diagram of FIG. 1B which illustrates how the initial treatment planning 30 may be performed and one or more initial aligners may be fabricated for use by the patient. After initial treatment, the patient may be assessed 32 and additional treatment options 34 may be generated or considered during additional treatment planning. Based on the assessment 32, various treatment options may be considered and the patient re-assessed 36, 38, for example, after a predetermined period of time. The assessment may be formed by the practitioner based on the progress or lack of progress of moving the patient's tooth or teeth to a desired position. Additionally, the patient may also collaborate with the practitioner to provide their own assessments, thoughts, etc. so that the practitioner may consider not only the physiological data but also the collaboration provided by the patient.

Depending on which treatment option was pursued, additional treatment options 40, 46 may be considered and their corresponding outcome re-assessed 42, 44, 48, 50 again depending on which treatment option was pursued. Depending upon the assessment and, if needed, the patient may again be provided with treatment options 52, 54, 56, 58 and the process may be continued at predetermined intervals until the desired outcome is reached. Because the treatment process is not predetermined from the start to the end of the entire treatment and the treatment options may be varied, the aligners may be fabricated with only a few at a time. This also provides flexibility to the practitioner to alter the treatment mid-course without having an entire array of pre-fabricated aligners un-used.

Returning to FIG. 1A, the fabrication process 20 itself may be accomplished through different methods. In one example, the model of the partially corrected patient dentition may be formed as a positive mold, e.g., via a 3D printing mold 22 and corresponding aligner or aligners may be thermal formed 24 upon the positive molds. In another example, the one or more aligners may be directly formed, e.g., direct 3D printing 26. In either case, the resulting one or more aligners 28 may be formed for use by the patient.

Obtaining a digital model of the patient's dentition for facilitating the treatment planning may be accomplished in a number of different ways. The patient may have their dentition scanned at another location and forwarded to the treatment provider or their dentition may be scanned directly at the treatment provider's location. In either case, the patient's dentition may be digitally scanned through any number of suitable scanning devices. For example, the patient may have their dentition (including teeth, soft tissue, or both) scanned by an MRI scanner, X-ray machine, intra-oral scanner, etc. The resulting scanned images may be saved or uploaded to a computer system and used to generate a digital image of the teeth which may be used for planning the treatment to correct for the positioning, misalignment, malocclusion, etc. of any one or more teeth. Alternatively, the patient's dentition may be cast to obtain an impression which may then be used to create a positive mold. The resulting positive mold reflecting the patient's dentition may then be scanned to obtain the corresponding digital image.

The treatment planning process may be implemented after receiving and analyzing the scanned dental model of a patient's dentition. The scanned dental model may be accordingly processed to enable the development of a treatment plan which can be readily implemented for fabricating one or more aligners for use in effecting sequential tooth movements.

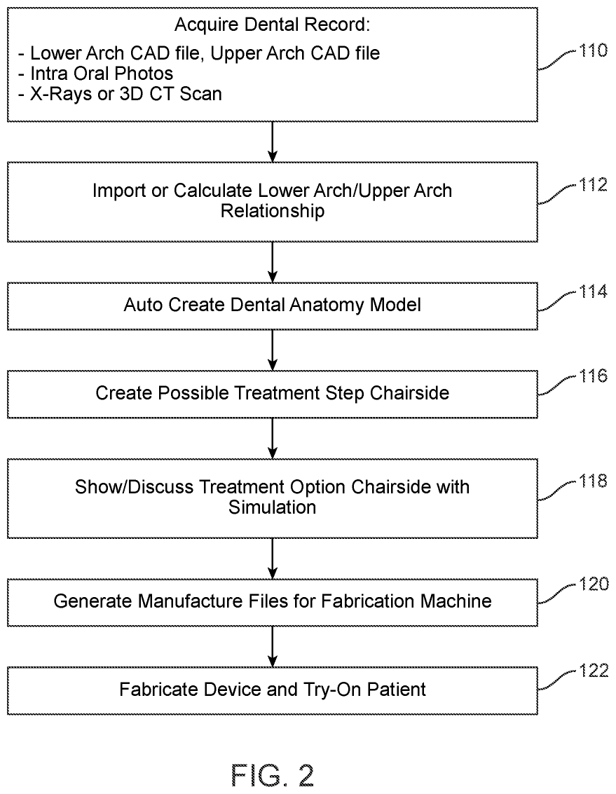

FIG. 2A shows an exemplary overall tooth modeling process which may be used in planning the treatment for correcting malocclusions in a patient. The process shown may involve initially acquiring a patient's dental record 110 in the form of, e.g., lower arch and/or upper arch CAD files, intra oral photos, X-rays or 3D CT scans, etc. The lower arch and/or upper arch CAD files may be created, for instance, through a number of different methods, such as taking lower and upper impressions of the patient's dentition, X-rays, etc.

Once the dental records are acquired, the lower arch and upper arch relationship may be imported or calculated 112 for registration by one or more computing devices and a flexible dental anatomy model may be auto created 114 by one or more processors located locally in proximity to where the patient is treated, e.g., dental office, or remotely from the patient location. Once the dental anatomy model has been digitally created and confirmed to fit and that the arch model can open and close as expected, one or more possible treatments may be created in real-time chairside of the patient 116 and the one or more treatment options may be shown and/or discussed with the patient chairside 118 where simulations of the treatment options may also be shown and/or discussed for potentially altering the treatment plan as needed. The simulations of treatment options may be displayed to the patient using any number of electronic display methods.

Following the discussion of the treatment options with the patient, the treatment plan (with any alterations) may be used to generate manufacturing files for the fabrication machinery 120, e.g., 3D printing machines, thermal forming, laser sintering, etc. Because the resulting one or more aligners may be fabricated locally in proximity to the patient (e.g., dental office, clinic, nearby facility, etc.) the one or more resulting aligners for use by the patient may be fabricated locally allowing for the patient to try on the one or more aligners 122 during the same visit.

Such a treatment plan may have particular advantages over conventional planning and treatment plans including one or more of the following:

exact treatment may be developed right way and discussed with the patient in real time;

practitioner has full control of the treatment plan options which are easy to create;

real gum modeling may be implemented;

one or more aligners may be fabricated locally allowing the patient to try-on during the same visit;

easy to incorporate other treatment methods, e.g. indirect bonding bracket, rubber bands, hooks, retainers, etc. in combination with one or more aligners.

Even in the event that a treatment plan has been developed and implemented for a patient, as shown and described for FIG. 2, the actual progress of the tooth movement(s) may not correspond to the treatment plan or the actual progress may begin to deviate from the treatment plan. Because of this variability, not all of the aligners or aligners may be fabricated at the start of the treatment but the aligners may instead be fabricated in preset stages for use by the patient until a subsequent visit to the practitioner, e.g., every six weeks, where a new set of aligners may be fabricated for subsequent treatments.

As will be apparent, the features and attributes of the specific embodiments disclosed herein may be combined in different ways to form additional embodiments, all of which fall within the scope of the present disclosure.

In preparing the treatment planning 18, the practitioner may prepare the treatment plan for correcting the malocclusions on their own but in other variations, the practitioner may collaborate with a treatment plan designer/technician trained in facilitating the treatment planning with the practitioner. However, collaborating over conventional communication modalities may be inefficient for various reasons. For instance, communicating over conventional teleconferencing software may prove inefficient due to the difficulty in sharing or communicating changes to a treatment plan or asynchronous methods such as email, text, etc. may also prove inefficient due to the lag in time and inability to collaborate in real-time. Hence, a software structure which is able to join the practitioner and the treatment plan designer/technician in a treatment planning environment may be incorporated into the treatment planning software to enable efficient collaboration in real-time. The treatment planning software, as described herein, may be implemented upon various devices or interfaces having a processor such as a computer, tablet, smartphone, etc.

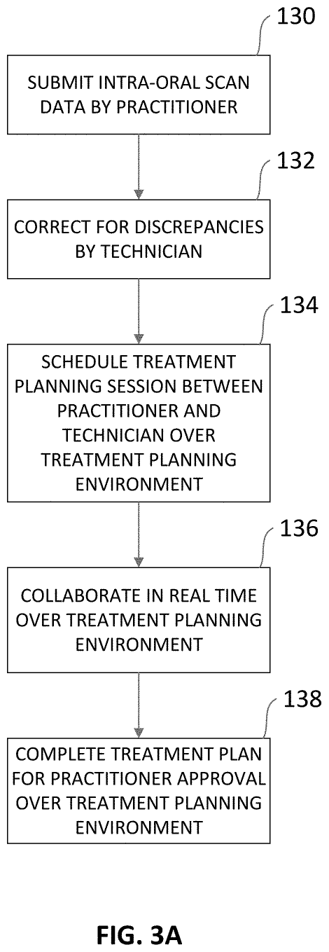

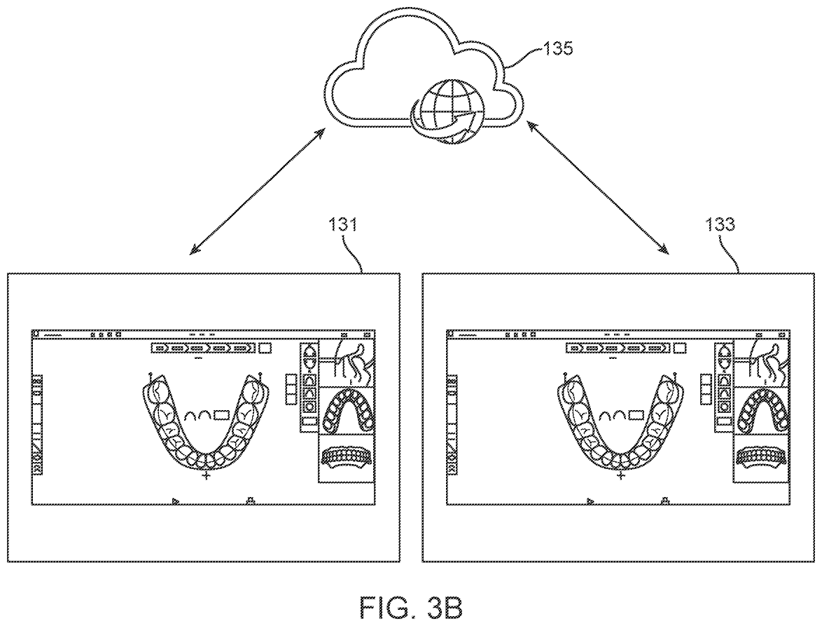

As shown in FIG. 3A, a flow diagram generally illustrates how the practitioner is able to collaborate in real-time with the treatment plan designer/technician through a treatment planning environment (TPE) module integrated into the treatment planning software. While the practitioner is logged into the TPE of the treatment planning software, the practitioner may electronically submit intra-oral scan data 130 of the patient's dentition, as described herein, obtained by the practitioner or another party. This intra-oral scan data may be uploaded to the TPE where the intra-oral scan data may be accessed by the treatment plan designer/technician (either in real-time simultaneously with the practitioner or at a later time) who may correct for any discrepancies 132 in the dentition by forming an initial treatment plan for review by the practitioner. The treatment plan designer/technician may process the scan data using the treatment planning software to perform various other operations such as patching holes that may be present in the scan file, fixing a bite between the upper and lower teeth, segmenting the teeth in the scan data, identifying features such as the FACC line and ridge line, etc. Once the treatment plan designer/technician has completed correcting the scan data, the treatment plan designer/technician may save the revised scan data which is then saved on the practitioner's computer as well.

The types of treatments to be planned within the TPE are not limited to any particular appliance but may vary from the application of aligners to any number of other orthodontic treatments. For example, the TPE treatment planning may implement the fabrication and/or use of other appliances such as indirect bonding trays, brackets, archwires, attachments, etc.

In the event that the treatment plan designer/technician accesses and corrects for discrepancies asynchronously from the practitioner, both the treatment plan designer/technician and practitioner may schedule a treatment planning session between the two over the TPE to discuss the initial treatment plan 134. One or more available time slots may appear on the TPE for both the treatment plan designer/technician and practitioner allowing for both parties to schedule a common time slot.

At the scheduled time, both the treatment plan designer/technician and practitioner may access the TPE simultaneously within the treatment planning software and collaborate in real-time over the TPE 136 to adjust any number of aspects of the treatment plan, as illustrated schematically in FIG. 3B. The treatment plan designer/technician or practitioner may host the session in the TPE, as illustrated upon the screens 131, 133 shown in the figure and in the case where the treatment plan designer/technician hosts, they may initiate the display of the data including the arches of the patient's dentition. The practitioner may instruct the treatment plan designer/technician how to set up the arches for the patient's final positioning for their teeth. The collaboration may include not only a display of the scan data upon the treatment plan designer/technician's screen 131 the practitioner's screen of their local computing device 133, but may also include voice communication over the TPE and optionally video communication of the treatment plan designer/technician and practitioner as well which may be transmitted through the internet and/or cloud storage service 135, as shown schematically. As the practitioner instructs the treatment plan designer/technician, the treatment plan designer/technician may implement the desired changes and alterations using the treatment planning software in real-time showing the changes reflected to both screens of the respective computing devices 131, 133 of the treatment plan designer/technician and practitioner within the TPE, illustrated upon the screens, so that the practitioner may visually see exactly what changes are being made to the digital model of the dentition for the treatment plan.

Once the treatment plan has been suitably adjusted in real time, the practitioner may approve the final treatment plan over the TPE 138 using an approval processes (such as pressing of a button in lieu of a signature) and the final treatment plan may be implemented by the practitioner upon the patient's dentition including the ordering of any orthodontic prostheses used to carry out the treatment plan. The TPE may then be closed at the end of this process.

While the example discussed the collaboration between the practitioner and treatment plan designer/technician, the number of practitioners and/or treatment plan designers/technicians is unlimited as any number of practitioners may collaborate within the TPE with any number of designers/technicians. Alternatively, any number of practitioners may collaborate with one another within the TPE without the presence of any designers/technicians and/or any number of designers/technicians may collaborate with one another within the TPE without the presence of any practitioners.

There are additional functions which may be optionally incorporated. In another variation, after the treatment plan has been approved, the practitioner can choose to send a link to the patient (e.g., via email, notification through an app on their tablet or smartphone, or through another communication method). Once the patient receives this link, they may access the results of the treatment plan showing, for instance, the movement of teeth from the initial position to a final position.

Additionally and/or alternatively, the TPE may be developed as a platform utilizing artificial intelligence algorithms to train the computers on the back end to understand the commands (verbal or textual) of the practitioner to implement their clinical demands so that the computer itself may implement any changes rather than a treatment plan designer/technician.

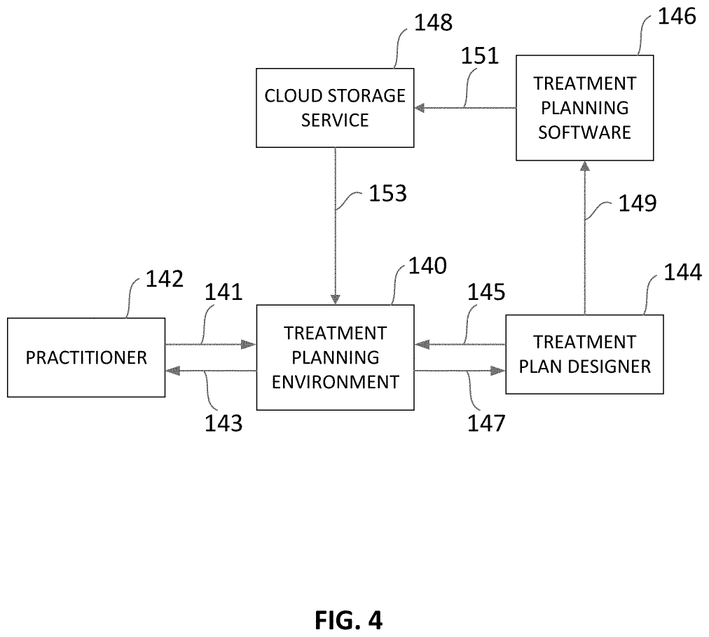

FIG. 4 shows a schematic diagram illustrating further details of how the practitioner and treatment plan designer/technician can facilitate the real-time collaboration through the TPE. As described, the TPE 140 may reside as a virtual module which is integrated with the treatment planning software. The practitioner 142 may interact with the TPE 140 through the internet and/or cloud storage service 135 and via their local computing device 133 with textual or voice feedback 141 in response to 3D visual feedback and/or voice feedback 143 from the treatment plan designer/technician 144 who may similarly provide voice feedback 145 to the practitioner 142 via the TPE 140 as well as receive the voice feedback 147 from the practitioner 142 via the TPE 140, as shown.

The treatment plan designer/technician 144 may implement any number of treatment planning operations 149 using the treatment planning software 146 in response to the feedback from the practitioner 142 received via the TPE 140. The treatment planning data as modified by the planning operations 149 may be transmitted 151 to the internet or cloud storage service 148 which may then transmit the information 153 through the TPE 140 for presentation to the practitioner 142 or directly to a local computing device of the practitioner 142 for review.

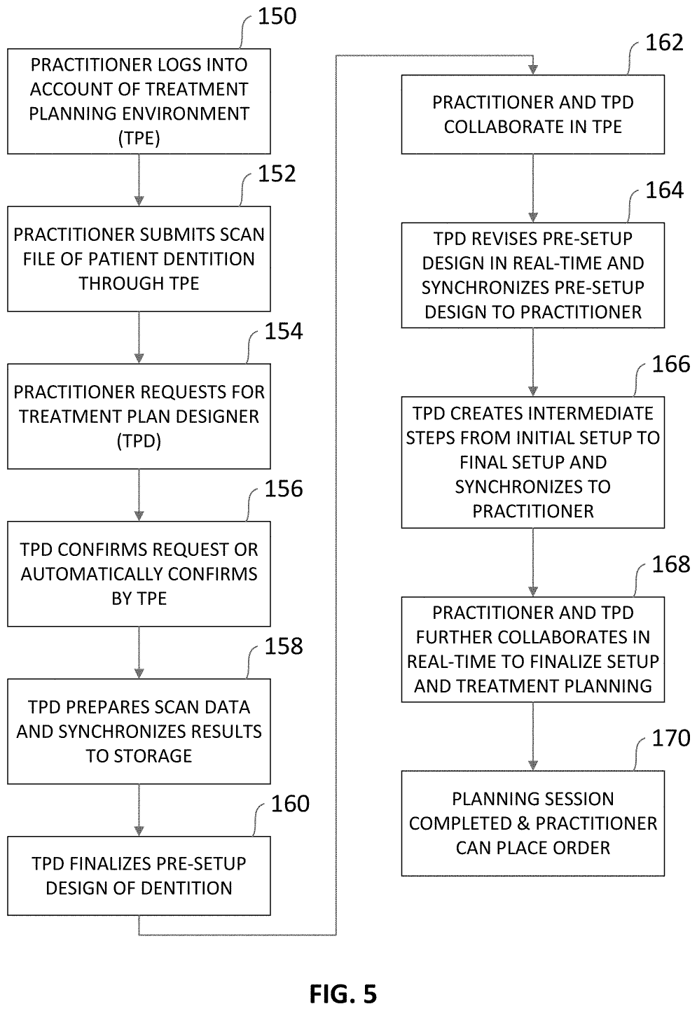

Another variation of the workflow is illustrated in the flow diagram shown in FIG. 5. The practitioner may log into their account of the TPE 150 to submit a scan file of the patient's dentition through the TPE 152. The practitioner may request for time with a treatment plan designer/technician 154 to guide the treatment plan by scheduling a time slot with the treatment plan designer/technician. The treatment plan designer/technician may confirm the request from the practitioner or the TPE may automatically confirm the request to the practitioner 156.

The treatment plan designer/technician may prepare the scan data submitted by the practitioner and synchronize the results to the cloud storage service 158. As described, the treatment plan designer/technician may prepare the scan data by correcting a number of features such as segmenting the scan data into teeth, defining the tooth ID, identifying anatomical features using the treatment planning software, positioning teeth in their final positions, etc. The treatment plan designer/technician may finalize the corrected scan data as a pre-setup design of the dentition for treatment planning 160.

At the scheduled time, both the practitioner and treatment plan designer/technician may meet virtually in the TPE environment to collaborate 162. During this time, the practitioner may provide guidance in real-time to the treatment plan designer/technician to revise the pre-setup design which may be synchronized with the results presented to the practitioner 164. During the collaboration, the practitioner may voice their opinion on the final setup of the dentition and the treatment plan designer/technician may modify the pre-setup accordingly using the treatment planning software. The treatment plan designer/technician may create intermediate steps from the initial setup to the final setup and again synchronize the results to the practitioner via the cloud storage service 166. The treatment plan designer/technician may create the intermediate steps by applying features such as attachments to the appropriate teeth, adjusting Interproximal Reduction (IPR) steps using the treatment planning software, etc. The practitioner may review the revised treatment plan in real-time and further collaborate with the treatment plan designer/technician to finalize a setup and treatment plan 168. Once the practitioner agrees on the final setup and the treatment plan, the planning session may be completed 170. The practitioner may optionally place an order or schedule a time to place an order at a later date for any number of orthodontic prostheses, if desired.

The system or method described herein may be deployed in part or in whole through a computer system or machine having one or more processors that execute software programs with the methods as described herein. The software programs may be executed on computer systems such as a server, domain server, Internet server, intranet server, and other variants such as secondary server, host server, distributed server, or other such computer or networking hardware on a processor. The processor may be a part of a server, client, network infrastructure, mobile computing platform, stationary computing platform, or other computing platform. The processor may be any kind of computational or processing device capable of executing program instructions, codes, binary instructions or the like that may directly or indirectly facilitate execution of program code or program instructions stored thereon. In addition, other devices required for execution of methods as described in this application may be considered as a part of the infrastructure associated with the computer system or server.

The system or method described herein may be deployed in part or in whole through network infrastructures. The network infrastructure may include elements such as computing devices, servers, routers, hubs, firewalls, clients, wireless communication devices, personal computers, communication devices, routing devices, and other active and passive devices, modules or components as known in the art. The computing or non-computing device(s) associated with the network infrastructure may include, apart from other components, a storage medium such as flash memory, buffer, stack, RAM, ROM, or the like. The processes, methods, program codes, and instructions described herein and elsewhere may be executed by the one or more network infrastructural elements.

The elements described and depicted herein, including flow charts, sequence diagrams, and other diagrams throughout the figures, imply logical boundaries between the elements. However, according to software or hardware engineering practices, the depicted elements and the functions thereof may be implemented on machines through the computer executable media having a processor capable of executing program instructions stored thereon and all such implementations may be within the scope of this document. Thus, while the foregoing drawings and descriptions set forth functional aspects of the disclosed methods, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this document. As such, the depiction or description of an order for various steps should not be understood to require a particular order of execution for those steps, unless required by a particular application, or explicitly stated or otherwise clear from the context.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device, or other hardware. All such permutations and combinations are intended to fall within the scope of the present disclosure.

The applications of the devices and methods discussed above are not limited to the dental applications but may include any number of further treatment applications. Moreover, such devices and methods may be applied to other treatment sites within the body. Modification of the above-described assemblies and methods for carrying out the invention, combinations between different variations as practicable, and variations of aspects of the invention that are obvious to those of skill in the art are intended to be within the scope of the claims.

What is claimed is:

1. A method of collaborating on a treatment plan for correcting one or more teeth of a subject, comprising:
digitally receiving intra-oral scan data of one or more teeth of the subject where the one or more teeth are in an initial position;
synchronizing a virtual treatment planning environment between a first device of at least one practitioner and a second device of at least one technician;
displaying a pre-setup design of the intra-oral scan data within the treatment planning environment, wherein the pre-setup design incorporates one or more corrections of the intra-oral scan data;
revising the pre-setup design based upon input received from the at least one practitioner within the treatment planning environment for correcting one or more malocclusions;
creating one or more intermediate steps of the treatment plan from the initial position to a final position of the one or more teeth within the treatment planning environment; and
finalizing the treatment plan by the practitioner within the treatment planning environment.

2. The method of claim 1 wherein digitally receiving the intra-oral scan data comprises receiving the intra-oral scan data to the treatment planning environment.

3. The method of claim 1 further comprising receiving a request for a collaboration session with the at least one technician from the at least one practitioner prior to synchronizing the virtual treatment planning environment.

4. The method of claim 3 further comprising confirming the request via the treatment planning environment.

5. The method of claim 1 further comprising preparing the pre-setup design by the at least one technician prior to synchronizing the virtual treatment planning environment.

6. The method of claim 5 wherein preparing the pre-setup design comprises correcting for one or more discrepancies in the intra-oral scan data.

7. The method of claim 1 wherein synchronizing the virtual treatment planning environment comprises synchronizing via a cloud storage service.

8. The method of claim 1 wherein displaying the pre-setup design comprises displaying the pre-setup upon the first device and the second device.

9. The method of claim 1 wherein revising the pre-setup design comprises revising the pre-setup design via a treatment planning software within the treatment planning environment.

10. The method of claim 9 further comprising receiving auditory input from the at least one practitioner via the treatment planning environment.

11. The method of claim 9 further comprising receiving auditory input from the at least one technician via the treatment planning environment.

12. The method of claim 9 wherein creating one or more intermediate steps comprises creating via the treatment planning software.

13. The method of claim 1 wherein finalizing the treatment plan comprises receiving approval from the at least one practitioner via the treatment planning environment.

14. The method of claim 1 further comprising receiving an order via the treatment planning environment from the practitioner for one or more orthodontic appliances.

15. A method for collaborating on a treatment plan for correcting one or more teeth of a subject, comprising:
digitally receiving intra-oral scan data of one or more teeth of the subject from at least one practitioner, wherein the one or more teeth are in an initial position;
confirming a request received from the at least one practitioner via a virtual treatment planning environment to collaborate with at least one technician;
synchronizing a pre-setup design of the intra-oral scan data with a first device of the practitioner with a second device of the at least one technician, wherein the pre-setup design incorporates one or more corrections provided by the at least one technician of the intra-oral scan data;
displaying the pre-setup design of the intra-oral scan data within the treatment planning environment to the at least one practitioner and the at least one technician;
revising the pre-setup design based upon input received from the at least one practitioner within the treatment planning environment for correcting one or more malocclusions; and
creating one or more intermediate steps of the treatment plan from the initial position to a final position of the one or more teeth within the treatment planning environment to complete the treatment plan.

16. The method of claim 15 further comprising finalizing the treatment plan by the at least one practitioner within the treatment planning environment.

17. The method of claim 15 wherein digitally receiving the intra-oral scan data comprises receiving the intra-oral scan data to the treatment planning environment.

18. The method of claim 15 wherein confirming the request further comprises receiving the request for a collaboration session from the at least one practitioner prior to confirming the virtual treatment planning environment.

19. The method of claim 15 wherein synchronizing the pre-setup design comprises synchronizing via a cloud storage service.

20. The method of claim 15 wherein displaying the pre-setup design comprises displaying the pre-setup upon the first device and the second device.

21. The method of claim 15 wherein revising the pre-setup design comprises revising the pre-setup design via a treatment planning software within the treatment planning environment.

22. The method of claim 15 wherein revising the pre-setup design comprises receiving auditory input from the at least one practitioner via the treatment planning environment.

23. The method of claim 22 further comprising receiving auditory input from the at least one technician via the treatment planning environment.

24. The method of claim 15 wherein creating one or more intermediate steps comprises creating via a treatment planning software.

25. The method of claim 16 wherein finalizing the treatment plan comprises receiving approval from the at least one practitioner via the treatment planning environment.

26. The method of claim 15 further comprising receiving an order via the treatment planning environment from the practitioner for one or more orthodontic appliances.
